(19) United States
(12) Patent Application Publication
(10) Pub. No.: US 2004/0204756 A1
(43) Pub. Date: Oct. 14, 2004

(54) ABSORBENT ARTICLE WITH IMPROVED LIQUID ACQUISITION CAPACITY

(76) Inventors: Stephen Hunter Diaz, Palo Alto, CA (US); Theodore L Parker, Danville, CA (US); John F Shanley, Redwood City, CA (US); Brett Trauthen, Newport Beach, CA (US); Frank Litvack, Los Angeles, CA (US)

Correspondence Address: BURNS DOANE SWECKER & MATHIS L L P, POST OFFICE BOX 1404, ALEXANDRIA, VA, 22313-1404, US

(21) Appl. No.: 10/777,881

(22) Filed: Feb. 11, 2004

(51) Int. Cl. A61F 2/06

(52) U.S. Cl. 623/1.42

(57) ABSTRACT

### Abstract of the Disclosure

A method for decreasing the level of restenosis following a stent placement medical intervention involves the continuous administration of a dose of an anti-restenotic agent , such as paclitaxel, from the stent to vascular tissue in need of treatment in a controlled, extended ,and substantially linear drug release profile. The method of substantially linear extended release increases the therapeutic effectiveness of administration of a given dosage. In one example, a method of reducing restenosis includes delivering paclitaxel from a stent to an artery at a minimum release rate of 1 percent of the total dosage of paclitaxel on the stent per day throughout an entire administration period from the time of implantation of the stent until the time that substantially all the paclitaxel is released from the stent.

### Detailed Description of the Invention

### Cross Reference to Related Applications

This application is a Continuation-in-Part of U.S. Patent Application Serial No. 10/447,587 filed May 28, 2003, which is incorporated herein by reference in its entirety.

### Background of Invention

Most coronary artery-related deaths are caused by atherosclerotic lesions which limit or obstruct coronary blood flow to heart tissue. To address coronary artery disease, doctors often resort to percutaneous transluminal coronary angioplasty (PTCA) or coronary artery bypass graft (CABG). PTCA is a procedure in which a small balloon catheter is passed down a narrowed coronary artery and then expanded to re-open the artery. The major advantage of angioplasty is that patients in which the procedure is successful need not undergo the more invasive surgical procedure of coronary artery bypass graft. A major difficulty with PTCA is the problem of post-angioplasty closure of the vessel, both immediately after PTCA (acute reocclusion) and in the long term (restenosis).

Coronary stents are typically used in combination with PTCA to reduce reocclusion of the artery. Stents are introduced percutaneously, and transported transluminally until positioned at a desired location. These devices are then expanded either mechanically, such as by the expansion of a mandrel or balloon positioned inside the device, or expand themselves by releasing stored energy upon actuation within the body. Once expanded within the lumen, these devices, called stents, become encapsulated within the body tissue and remain a permanent implant.

Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen. Despite the introduction of improved surgical techniques, devices, and pharmaceutical agents, the overall restenosis rate is still reported in the range of 25% to 50% within six to twelve months after an angioplasty procedure. To treat this condition, additional revascularization procedures are frequently required, thereby increasing trauma and risk to the patient.

While the exact mechanisms of restenosis are still being determined, certain agents have been demonstrated to reduce restenosis in humans. One example of an agent which has been demonstrated to reduce restenosis when delivered from a stent is paclitaxel, a well-known compound that is commonly used in the treatment of cancerous tumors. However, many of the stents which are currently under development for delivery of anti-restenotic agents have suboptimal agent release profiles and side effects. In one example, over 90 % of the total agent loaded onto the stent is permanently retained in a thin coating on the surface of the stent and is never delivered to the tissue.

### Summary of Invention

The present invention relates to a method for decreasing restenosis following stenting by administration of an anti-restenotic agent in a controlled drug release profile which increases the therapeutic effectiveness of administration. The present invention also relates to a stent having a dosage of anti-restenotic agent affixed thereto for controlled release of the agent at a programmed drug delivery profile.

In accordance with one aspect of the invention, a method of reducing restenosis is provided, wherein the method involves providing a drug delivery stent having a dosage of paclitaxel for delivery to an artery, the dosage arranged such that substantially all the paclitaxel is releasable from the stent upon implantation of the stent in the artery. The method further involves implanting the stent within an artery of a patient; and delivering paclitaxel from the stent to the artery at a minimum release rate of 1 percent of the total dosage of paclitaxel on the stent per day throughout an entire administration period from the time of implantation of the stent until the time that substantially all the paclitaxel is released from the stent.

In accordance with another aspect of the invention, a method of reducing restenosis is provided, wherein the method involves providing a drug delivery stent having a dosage of paclitaxel for delivery to an artery. The method further involves implanting the stent within an artery of a patient; and delivering paclitaxel from the stent to the artery at a substantially linear release rate over an entire period from day one after implantation through day twenty five after implantation, wherein the amount of paclitaxel delivered during the period is at least 25% of the drug loaded on the stent.

In accordance with an additional aspect of the invention, a method of reducing restenosis is provided, wherein the method involves providing a drug delivery stent having a dosage of paclitaxel for delivery to an artery. The method further involves implanting the stent within an artery of a patient; and delivering paclitaxel from the stent to the artery, wherein at least 80% of the entire dosage of paclitaxel provided by the stent is delivered to the artery within 60 days of implantation.

In accordance with a further aspect of the invention, a method of reducing restenosis is provided, wherein the method involves a drug delivery stent having a dosage of an anti-restenotic drug for delivery to an artery, the dosage arranged such that substantially all the drug is releasable from the stent upon implantation of the stent in the artery. The method further involves implanting the stent within an artery of a patient; and delivering the drug from the stent to the artery at a minimum release rate of 1 percent of the total dosage of the drug on the stent per day throughout an entire administration period from the time of implantation of the stent until the time that substantially all the drug is released from the stent, wherein the release rate of the drug is substantially linear from at least day two through day 25.

In accordance with a further aspect of the invention, a method of treating a patient is provided, wherein the method involves providing a drug delivery stent having a dosage of therapeutic agent for delivery to an artery, the dosage arranged such that substantially all the agent is releasable from the stent upon implantation of the stent in the artery. The method further involves implanting the stent within an artery of a patient; and delivering the agent from the stent to the artery at a minimum release rate of 1 percent of the total dosage of the agent on the stent per day throughout an entire administration period from the time of implantation of the stent until the time that substantially all the drug is released from the stent, wherein the release rate of the drug after day one is substantially linear from at least day 2 through day 25.

In accordance with a further aspect of the invention, a stent for reducing restenosis is provided, wherein the stent includes a drug delivery stent having initial unexpanded diameter for insertion of the stent into a coronary artery and an expanded diameter for implantation within a coronary artery. The stent further includes a dosage of paclitaxel for delivery to an artery, the dosage arranged such that substantially all the paclitaxel is releasable from the stent upon implantation of the stent in the artery. Furthermore, the dosage of paclitaxel is arranged to be released at a minimum release rate of 1 percent of the total dosage of paclitaxel on the stent per day throughout an entire administration period from the time of implantation of the stent until the time that substantially all the paclitaxel is released from the stent.

### Brief Description of Drawings

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1.

FIG. 3 is a side cross sectional view of an example of an opening in a stent showing a matrix with a therapeutic agent and a barrier layer.

FIG. 4 is a side cross sectional view of another example of an opening in a stent showing a matrix with a therapeutic agent.

FIG. 5 is a graph of the cumulative release of paclitaxel from a stent for three different substantially linear release profiles.

### Detailed Description

DETAILED DESCRIPTION

A method for decreasing the level of restenosis following a stent placement medical intervention involves the continuous administration of a dose of an anti-restenotic agent or drug from the stent to vascular tissue in need of treatment in a controlled, extended, and substantially linear drug release profile. It is envisioned that the vascular tissue in need of treatment is arterial tissue, specifically coronary arterial tissue. The method of substantially linear extended release increases the therapeutic effectiveness of administration of a given dose of anti-restenotic agent and reduces side effects.

In one example described in detail herein the agent or drug will be contained in reservoirs in the stent body prior to release. In the reservoir example, the drug will be held within the reservoirs in the stent in a drug delivery matrix comprised of the drug and a polymeric material and optionally additives to regulate the drug release. Preferably the polymeric material is a bioresorbable polymer.

The following terms, as used herein, shall have the following meanings:

The terms "drug" and "therapeutic agent" are used interchangeably to refer to any therapeutically active substance that is delivered to a living being to produce a desired, usually beneficial, effect.

The term "matrix" or "biocompatible matrix" are used interchangeably to refer to a medium or material that, upon implantation in a subject, does not elicit a detrimental response sufficient to result in the rejection of the matrix. The matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term "bioresorbable" refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, preferably less than one year, while maintaining any requisite structural integrity in that same time period.

The term openings includes both through openings and recesses.

The term pharmaceutically acceptable refers to the characteristic of being non-toxic to a host or patient and suitable for maintaining the stability of a therapeutic agent and allowing the delivery of the therapeutic agent to target cells or tissue.

The term "polymer" refers to molecules formed from the chemical union of two or more repeating units, called monomers. Accordingly, included within the term "polymer" may be, for example, dimers, trimers and oligomers. The polymer may be synthetic, naturallyor semisynthetic. In preferred form, the term "polymer" refers to molecules which typically have a Mw greater than about 3000 and preferably greater than about 10,000 and a Mw that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term primarily with respect to directional delivery, refers to an amount greater than about 50% of the total amount of therapeutic agent provided to a blood vessel.

The term restenosis refers to the renarrowing of an artery following an angioplasty procedure which may include stenosis following stent implantation.

The term substantially linear release profile refers to a release profile defined by a plot of the cumulative drug released versus the time during which the release takes place in which the linear least squares fit of such a release profile plot has a correlation coefficient, r² (the square of the correlation coefficient of the least squares regression line), of greater than 0.92 for data time points after the first day of delivery. A substantially linear release profile is clinically significant in that it allows release of a prescribed dosage of drug at a uniform rate over an administration period. This controlled release can be essential to staying within the toxic / therapeutic window for a particular drug.

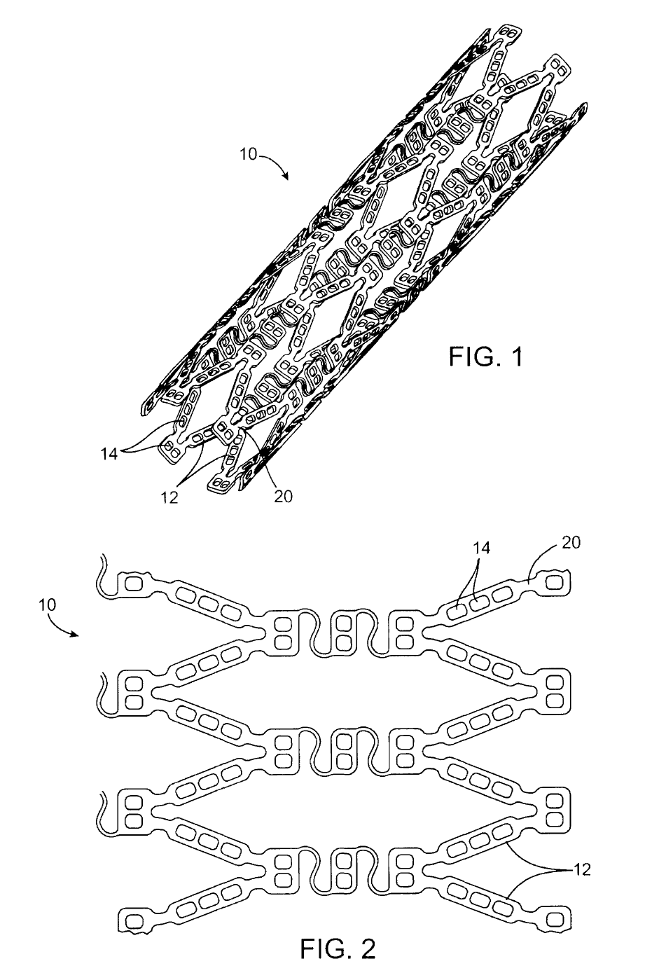

FIG. 1 illustrates one example of an implantable medical device in the form of a stent 10. FIG. 2 is an enlarged flattened view of a portion of the stent of FIG. 1 illustrating one example of a stent structure including struts 12 interconnected by ductile hinges 20. The struts 12 include openings 14 which can be non-deforming openings containing a therapeutic agent. One example of a stent structure having non-deforming openings is shown in U.S. Patent No. 6,562,065 which is incorporated herein by reference in its entirety.

The implantable medical devices of the present invention are configured to release at least one therapeutic agent from a matrix affixed to the implantable body. The matrix is formed such that the distribution of the agent in the polymer matrix directly controls the rate of elution of the agent from the matrix.

In one embodiment, the matrix is a polymeric material which acts as a binder or carrier to hold the agent in or on the stent and/or modulate the release of the agent from the stent. The polymeric material can be a bioresorbable or a non-bioresorbable material.

The therapeutic agent containing matrix can be disposed in the stent or on surfaces of the stent in various configurations, including within volumes defined by the stent, such as openings, holes, or concave surfaces, as a reservoir of agent, or arranged in or on all or a portion of surfaces of the stent structure. When the therapeutic agent matrix is disposed within openings in the strut structure of the stent to form a reservoir, the openings may be partially or completely filled with matrix containing the therapeutic agent.

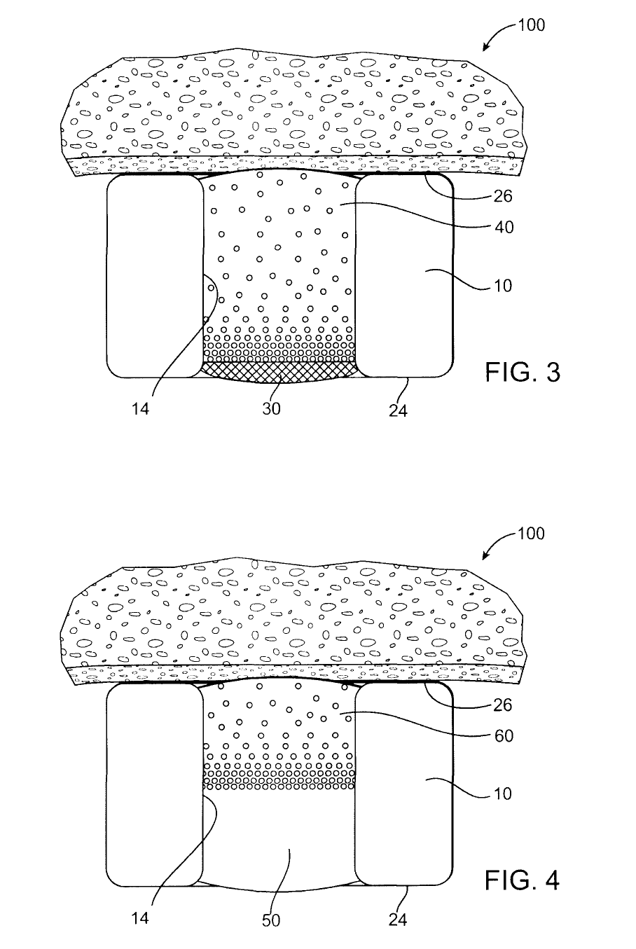

FIG. 3 is a cross section of one strut of the stent 10 and blood vessel 100 illustrating one example of an opening 14 arranged adjacent the vessel wall with a mural surface 26 abutting the vessel wall and a luminal surface 24 opposite the mural surface. The opening 14 of FIG. 3 contains a matrix 40 with a therapeutic agent illustrated by Os in the matrix. The luminal side 24 of the stent opening 14 is provided with a barrier layer 30. The barrier layer 30 erodes more slowly than the matrix 40 containing the therapeutic agent and thus, causes the therapeutic agent to be delivered primarily to the mural side 26 of the stent. The matrix 40 and therapeutic agent are arranged in a programmable manner to achieve a desire release rate and administration period which will be described in further detail below. As can be seen in the example of FIG. 3, the concentration of the therapeutic agent (Os) is highest at the luminal side 24 of the stent 10 and lowest at the mural side 26 of the stent. This configuration in which the drug can be precisely arranged within the matrix allows the release rate and administration period to be selected and programmed to a particular application. The methods by which the drug can be precisely arranged within the matrix in the openings is a stepwise deposition process is further described in U.S. Patent Application Serial No. _______ (Attorney Docket No. 032304-108) filed on even date herewith, and is incorporated herein by reference.

FIG. 4 is a cross section of another example of an opening 14 in a stent 10 containing a matrix and therapeutic agent. The opening 14 of FIG. 4 contains a matrix with a therapeutic agent illustrated by Os in the matrix. The portion of the matrix 50 located at the luminal ¼to ¾of the stent opening 14 includes matrix without the anti-restenotic agent while the portion of the matrix 60 located at the mural ¼to ¾ of the stent opening includes matrix with anti-restenotic agent. Preferably, the matrix with anti-retenotic agent 60 is located in about the mural ½ of the stent opening. An arrangement with the anti-restenotic agent positioned closer to the mural side 26 of the stent achieves directional delivery of the anti-restenotic agent primarily to the mural side with or without a barrier layer as described above. The matrix 50 portion and matrix and anti-restenotic agent 60 portion are arranged in a programmable manner to achieve a desire release rate and administration period which will be described in further detail below. As can be seen FIG. 4, the concentration of the therapeutic agent (Os) is highest at a center of the stent 10 and lower at the mural side 26 of the stent to achieve a substantially linear release rate with a minimal initial start up release.

Numerous other useful arrangements of the matrix and therapeutic agent can be formed to achieve the substantially linear release, extended release, and substantially complete release described herein. Each of the areas of the matrix may include one or more agents in the same or different proportions from one area to the next. The matrix may be solid, porous, or filled with other drugs or excipients. The agents may be homogeneously disposed or heterogeneously disposed in different areas of the matrix.

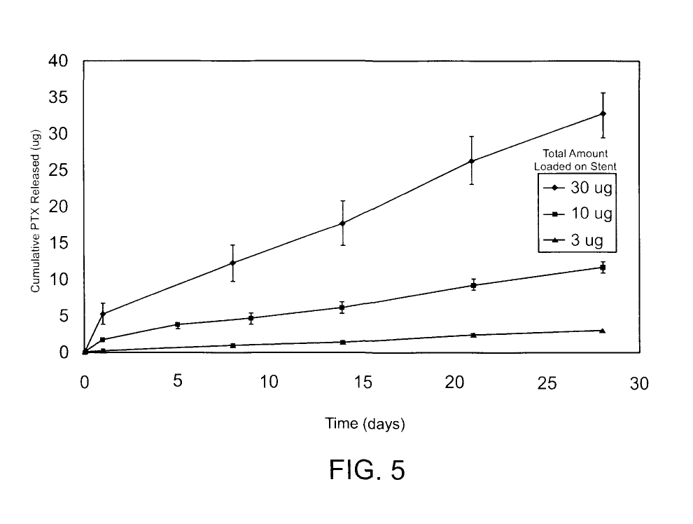

FIG. 5 illustrates three examples of extended-linear drug release profiles which are characterized by a small initial release of drug in the first day, followed by a substantially linear extended release until all the drug loaded on the stent is released. Preferably, the initial release in the first day of administration will be less than 25% of the total drug loaded. In the examples, the drug released is paclitaxel which is loaded in a PLGA matrix for directional delivery to the mural side of the stent. The drug release rate is programmed by providing different concentrations of drug in different areas of the matrix.

The method for administering a dose of anti-restenotic agent, such as paclitaxel, can include delivering 2-25% of the total amount of agent loaded into the stent in the first day, then delivering drug in a substantially linear fashion a total 95% of the loaded drug by day 20-45. Following the first day release, the rate of extended substantially linear drug release will be in the range of greater than 1% per day, preferably about 1.5% to about 5% of the total loaded drug dose per day, and more preferably the substantially linear release rate is in the range of about 2% to about 4% of total drug loaded per day.

The release profile for a drug or therapeutic agent can be defined by a plot of the cumulative drug released versus the time during which the release takes place, as shown in FIG. 4. By substantially linear release profile is meant that the linear least squares fit of such a release profile plot has a correlation coefficient value, r², of greater than 0.92 for data time points after the first day of delivery. According to one preferred embodiment, an anti-restenotic, such as paclitaxel is released at a substantially linear release rate in which r² is greater than 0.95 after the first day of delivery with less than 25% of the total drug loaded delivered in the first day.

When the anti-restenotic agent delivered by the method of the invention is paclitaxel, the total amount delivered (and loaded) is preferably between 2 micrograms and 50 micrograms. In one preferred embodiment, the amount of paclitaxel delivered will be between about 0.1 micrograms and about 15 micrograms on the first day, more preferably between about 0.3 micrograms and about 9 micrograms. Following day one, the paclitaxel will be delivered in a substantially linear fashion at a rate of about 0.025 micrograms to about 2.5 microgram per day for a minimum of 21 days, preferably about 0.2 to about 2 micrograms per day. It is envisioned that all the paclitaxel will be released from the stent in less than 60 days. The total amount of paclitaxel loaded onto the stent and released into the tissue in need of treatment is preferably in the range of about 1.5 micrograms to about 75 micrograms, more preferably about 3 to about 30 micrograms. The above release rates for paclitaxel have been given for a standard stent of dimensions 3.0 mm in expanded diameter by 17 mm in length. Stents of other dimensions will contain total drug loadings in similar respective proportions based on similar drug loading density. In one example, the amount of paclitaxel released per day after day one is about 0.0003 to about 0.03 ug/mm² of tissue surface area, preferably about 0.0003 to about 0.01 ug/mm² of tissue surface area. In another example, the amount of paclitaxel released per day after day one is about 0.001 to about 0.2 ug/mm of stent length per day.

The methods of the invention preferably will result in sustained release of substantially all the drug loaded onto the stent in no longer than 180 days, preferably in no longer than 60 days, and most preferably in no longer than 35 days.

When the anti-restenotic agent is paclitaxel, at least 50% of the paclitaxel loaded into the stent is preferably released and no more than 50% of the amount is non-releasable. Non-releasable paclitaxel is paclitaxel that is sequestered in the polymeric matrix such that it is not released under physiologic conditions is less than 180 days. Preferably, more than 80% of the paclitaxel loaded will be released in no longer than 180 days, more preferably all the paclitaxel will be released.

In one preferred embodiment, agent will be delivered from a polymer matrix reservoir in the stent, where the polymer is a bioresorbable polymer. In the case of a bioresorbable polymer, preferably all of the drug is eluted from the stent before all of the polymer matrix is resorbed. Typically all polymer drug delivery matrix will be bioresorbed in 14 days to one year, more preferably in 30 days to 90 days.

The substantially linear extended drug delivery profiles described above and the examples shown in FIG. 5 can become a zero order release profile, or can be a zero order release profile after the second day of drug delivery.

It has been shown in clinical trials that longer constant or substantially linear release of the anti-restenotic paclitaxel, such as in the release profiles shown in FIG. 5 results in lower in stent neointimal proliferation than the more rapid release of the same dosage. The method of substantially linear extended release of anti-restenotic agents increases the therapeutic effectiveness of administration of a given dose of agent and reduces side effects.

While the invention has been describe with respect to treatment of restenosis, other therapeutic agents may be delivered at the release profiles described for treatment of acute myocardial infarction, thrombosis, or for passivation of vulnerable plaque.

THERAPEUTIC AGENTS

The present invention relates to the delivery of anti-restenotic agents including taxol, rapamycin, cladribine, colchicines, vinca alkaloids, heparin, hinrudin and their derivatives, as well as other cytotoxic or cytostatic agents and microtubule stabilizing and microtubule inhibiting agents. Although anti-restenotic agents have been primarily described herein, the present invention may also be used to deliver other agents alone or in combination with anti-restenotic agents. Some of the therapeutic agents for use with the present invention which may be transmitted primarily luminally, primarily murally, or both and may be delivered alone or in combination include, but are not limited to, antiproliferatives, antithrombins, immunosuppressants including sirolimus, antilipid agents, anti-inflammatory agents, antineoplastics, antiplatelets, angiogenic agents, anti-angiogenic agents, vitamins, antimitotics, metalloproteinase inhibitors, NO donors, estradiols, anti-sclerosing agents, and vasoactive agents, endothelial growth factors, estrogen, beta blockers, AZ blockers, hormones, statins, insulin growth factors, antioxidants, membrane stabilizing agents, calcium antagonists, retenoid, bivalirudin, phenoxodiol, etoposide, ticlopidine, dipyridamole, and trapidil alone or in combinations with any therapeutic agent mentioned herein. Therapeutic agents also include peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense, oligonucleotides, monoclonal antibodies, platelets, prions, viruses, bacteria, and eukaryotic cells such as endothelial cells, stem cells, ACE inhibitors, monocyte/macrophages or vascular smooth muscle cells to name but a few examples. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the therapeutic layer. Therapeutic agents may also be radioactive isotopes or agents activated by some other form of energy such as light or ultrasonic energy, or by other circulating molecules that can be systemically administered. Therapeutic agents may perform multiple functions including modulating angiogenesis, restenosis, cell proliferation, thrombosis, platelet aggregation, clotting, and vasodilation.

Anti-inflammatories include but are not limited to non-steroidal anti-inflammatories (NSAID), such as aryl acetic acid derivatives, e.g., Diclofenac; aryl propionic acid derivatives, e.g., Naproxen; and salicylic acid derivatives, e.g., Diflunisal. Anti-inflammatories also include glucocoriticoids (steroids) such as dexamethasone, aspirin, prednisolone, and triamcinolone, pirfenidone, meclofenamic acid, tranilast, and nonsteroidal anti-inflammatories. Anti-inflammatories may be used in combination with antiproliferatives to mitigate the reaction of the tissue to the antiproliferative.

The agents can also include anti-lymphocytes; anti-macrophage substances; immunomodulatory agents; cyclooxygenase inhibitors; anti-oxidants; cholesterol-lowering drugs; statins and angiotens in converting enzyme (ACE); fibrinolytics; inhibitors of the intrinsic coagulation cascade; antihyperlipoproteinemics; and anti-platelet agents; anti-metabolites, such as 2-chlorodeoxy adenosine (2-CdA or cladribine); immuno-suppressants including sirolimus, everolimus, tacrolimus, etoposide, and mitoxantrone; anti-leukocytes such as 2-CdA, IL-1 inhibitors, anti-CD116/CD18 monoclonal antibodies, monoclonal antibodies to VCAM or ICAM, zinc protoporphyrin; anti-macrophage substances such as drugs that elevate NO; cell sensitizers to insulin including glitazones; high density lipoproteins (HDL) and derivatives; and synthetic facsimile of HDL, such as lipator, lovestatin, pranastatin, atorvastatin, simvastatin, and statin derivatives; vasodilators, such as adenosine, and dipyridamole; nitric oxide donors; prostaglandins and their derivatives; anti-TNF compounds; hypertension drugs including Beta blockers, ACE inhibitors, and calcium channel blockers; vasoactive substances including vasoactive intestinal polypeptides (VIP); insulin; cell sensitizers to insulin including glitazones, P par agonists, and metformin; protein kinases; antisense oligonucleotides including resten-NG; antiplatelet agents including tirofiban, eptifibatide, and abciximab; cardio protectants including, VIP, pituitary adenylate cyclase-activating peptide (PACAP), apoA-I milano, amlodipine, nicorandil, cilostaxone, and thienopyridine; cyclooxygenase inhibitors including COX-1 and COX-2 inhibitors; and petidose inhibitors which increase glycolitic metabolism including omnipatrilat. Other drugs which may be used to treat inflammation include lipid lowering agents, estrogen and progestin, endothelin receptor agonists and interleukin-6 antagonists, and Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEF and PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

While the invention has been described in detail with reference to the preferred embodiments thereof, it will be apparent to one skilled in the art that various changes and modifications can be made and equivalents employed, without departing from the present invention.

What is Claimed is:

1. A method of reducing restenosis comprising:
providing a drug delivery stent having a dosage of paclitaxel for delivery to an artery, the dosage arranged such that substantially all the paclitaxel is releasable from the stent upon implantation of the stent in the artery;
implanting the stent within an artery of a patient; and
delivering paclitaxel from the stent to the artery at a minimum release rate of 1 percent of the total dosage of paclitaxel on the stent per day throughout an entire administration period from the time of implantation of the stent until the time that substantially all the paclitaxel is released from the stent.

2. The method of Claim 1, wherein the administration period is about 20 to about 40 days from the date of implantation.

3. The method of Claim 1, wherein the release profile of the paclitaxel after day one is substantially linear.

4. The method of Claim 1, wherein the amount of paclitaxel released per day after day one is about 0.0003 to about 0.03 ug/mm² of tissue surface area.

5. The method of Claim 1, wherein the paclitaxel is deposited in openings in the stent.

6. The method of Claim 1, wherein the paclitaxel is contained in a bioresorbable matrix.

7. The method of Claim 1, wherein the paclitaxel is contained in a polymer matrix.

8. The method of Claim 1, wherein the paclitaxel is delivered primarily murally from the stent.

9. The method of Claim 1, wherein the step of delivering paclitaxel further comprises delivering 2-25% of the total amount of paclitaxel loaded into the stent in the first day, then delivering 95% of the loaded paclitaxel by day 20 to 45.

10. The method of Claim 1, wherein the step of delivering paclitaxel further comprises delivering paclitaxel after day one at a rate of about 0.25 micrograms to about 2.5 microgram per day for a minimum of 21 days for a stent with dimensions 3.0 mm in expanded diameter by 17 mm in length, and delivering other amounts from stents of other dimensions based on their respective relative proportions.

11. The method of Claim 1, wherein the step of delivering paclitaxel further comprises delivering more than 80% of the paclitaxel loaded on the stent in no longer than 180 days.

12. A method of reducing restenosis comprising:
providing a drug delivery stent having a dosage of paclitaxel for delivery to an artery;
implanting the stent within an artery of a patient; and
delivering paclitaxel from the stent to the artery at a substantially linear release rate over an entire period from day one after implantation through day twenty five after implantation, wherein the amount of paclitaxel delivered during the period is at least 25% of the drug loaded on the stent.

13. The method of Claim 12, wherein the amount of paclitaxel released per day after day one is about 0.0003 to about 0.03 ug/mm² of tissue surface area.

14. The method of Claim 12, wherein the paclitaxel is deposited in openings in the stent.

15. The method of Claim 12, wherein the paclitaxel is contained in a bioresorbable polymer matrix.

16. The method of Claim 12, wherein the paclitaxel is delivered primarily murally from the stent.

17. The method of Claim 12, wherein the step of delivering paclitaxel further comprises delivering 2-25% of the total amount of paclitaxel loaded into the stent in the first day, then delivering 95% of the loaded paclitaxel by day 20 to 45.

18. The method of Claim 12, wherein the step of delivering paclitaxel further comprises delivering more than 80% of the paclitaxel loaded on the stent in no longer than 30 days.

19. A method of reducing restenosis comprising:
providing a drug delivery stent having a dosage of paclitaxel for delivery to an artery;
implanting the stent within an artery of a patient; and
delivering paclitaxel from the stent to the artery, wherein at least 80% of the entire dosage of paclitaxel provided by the stent is delivered to the artery within 60 days of implantation.

20. The method of Claim 19, wherein the release profile of the paclitaxel after day one is substantially linear.

21. The method of Claim 19, wherein the amount of paclitaxel released per day after day one is about 0.0003 to about 0.03 ug/mm² of tissue surface area.

22. The method of Claim 19, wherein the paclitaxel is deposited in openings in the stent.

23. The method of Claim 19, wherein the paclitaxel is contained in a bioresorbable polymer matrix.

24. The method of Claim 19, wherein the paclitaxel is delivered primarily murally from the stent.

25. The method of Claim 1, wherein the step of delivering paclitaxel further comprises delivering paclitaxel after day one at a rate of about 0.25 micrograms to about 2.5 microgram per day for a minimum of 21 days for a stent with dimensions 3.0 mm in expanded diameter by 17 mm in length, and delivering other amounts from stents of other dimensions based on their respective relative proportions.

26. A method of reducing restenosis comprising:
providing a drug delivery stent having a dosage of an anti-restenotic drug for delivery to an artery, the dosage arranged such that substantially all the drug is releasable from the stent upon implantation of the stent in the artery;
implanting the stent within an artery of a patient; and
delivering the drug from the stent to the artery at a minimum release rate of 1 percent of the total dosage of the drug on the stent per day throughout an entire administration period from the time of implantation of the stent until the time that substantially all the drug is released from the stent, wherein the release rate of the drug is substantially linear from at least day two through day 25.

27. The method of Claim 27, wherein the administration period is about 20 to about 40 days from the date of implantation.

28. The method of Claim 27, wherein the drug is deposited in openings in the stent.

29. The method of Claim 27, wherein the drug is contained in a bioresorbable polymer matrix.

30. The method of Claim 27, wherein the drug is delivered primarily murally from the stent.

31. The method of Claim 27, wherein the step of delivering drug further comprises delivering 2-25% of the total amount of drug loaded into the stent in the first day, then delivering 95% of the loaded drug by day 20 to 45.

32. The method of Claim 1, wherein the step of delivering drug further comprises delivering more than 80% of the drug loaded on the stent in no longer than 180 days.

33. The method of Claim 1, wherein the step of delivering drug further comprises releasing the drug at a substantially linear release rate in which r² is greater than 0.95 after the first day of delivery and with less than 25% of the total drug loaded delivered in the first day.

34. A method of treating a patient comprising:
providing a drug delivery stent having a dosage of therapeutic agent for delivery to an artery, the dosage arranged such that substantially all the agent is releasable from the stent upon implantation of the stent in the artery;
implanting the stent within an artery of a patient; and
delivering the agent from the stent to the artery at a minimum release rate of 1 percent of the total dosage of the agent on the stent per day throughout an entire administration period from the time of implantation of the stent until the time that substantially all the drug is released from the stent, wherein the release rate of the drug after day one is substantially linear from at least day 2 through day 25.

35. The method of Claim 35, wherein the administration period is about 20 to about 40 days from the date of implantation.

36. The method of Claim 35, wherein the drug is deposited in openings in the stent.

37. The method of Claim 35, wherein the drug is contained in a bioresorbable polymer matrix.

38. The method of Claim 35, wherein the step of delivering drug further comprises releasing the drug at a substantially linear release rate in which r² is greater than 0.95 after the first day of delivery and with less than 25% of the total drug loaded delivered in the first day.

39. A stent for reducing restenosis comprising:
a drug delivery stent having initial unexpanded diameter for insertion of the stent into a coronary artery and an expanded diameter for implantation within a coronary artery, the stent having a dosage of paclitaxel for delivery to an artery, the dosage arranged such that substantially all the paclitaxel is releasable from the stent upon implantation of the stent in the artery, wherein the dosage of paclitaxel is arranged to be released at a minimum release rate of 1 percent of the total dosage of paclitaxel on the stent per day throughout an entire administration period from the time of implantation of the stent until the time that substantially all the paclitaxel is released from the stent.

40. The stent of Claim 40, wherein the administration period is about 20 to about 40 days from the date of implantation.

41. The stent of Claim 40, wherein the release rate of the paclitaxel after day one is substantially linear.

42. The stent of Claim 40, wherein the amount of paclitaxel released per day after day one is about 0.0003 to about 0.03 ug/mm² of tissue surface area.

43. The stent of Claim 40, wherein the paclitaxel is deposited in openings in the stent.

44. The stent of Claim 40, wherein the paclitaxel is contained in a bioresorbable matrix.

45. The stent of Claim 40, wherein the paclitaxel is contained in a polymer matrix.

46. The stent of Claim 40, wherein the paclitaxel is arranged to be delivered primarily murally from the stent.

47. The stent of Claim 40, wherein the paclitaxel is affixed to the stent such that 2-25% of the total amount of paclitaxel loaded into the stent is delivered in the first day, 95% of the loaded paclitaxel delivered by day 20 to 45.

48. The stent of Claim 40, wherein the paclitaxel is loaded for delivery after day one at a rate of about 0.25 micrograms to about 2.5 microgram per day for a minimum of 21 days for a stent with dimensions 3.0 mm in expanded diameter by 17 mm in length, and delivering other amounts from stents of other dimensions based on their respective relative proportions.

49. The stent of Claim 40, wherein the paclitaxel is affixed to the stent such that more than 80% of the paclitaxel loaded on the stent is delivered in no longer than 180 days.
